(19) United States
(12) Patent Application Publication
(10) Pub. No.: US 2014/0207027 A1
(43) Pub. Date: Jul. 24, 2014

(54) WOUND COVER APPARATUS AND METHOD

(71) Applicant: THE CLEVELAND CLINIC FOUNDATION, Cleveland, OH (US)

(72) Inventors: Jose L. Navia, Shaker Hts., OH (US); Shengqiang Gao, Beachwood, OH (US)

(73) Assignee: THE CLEVELAND CLINIC FOUNDATION, Cleveland, OH (US)

(21) Appl. No.: 14/161,908

(22) Filed: Jan. 23, 2014

Related U.S. Application Data
(60) Provisional application No. 61/756,005, filed on Jan. 24, 2013.

(51) Int. Cl. A61F 13/00; A61H 7/00; A61M 1/00

(52) U.S. Cl. CPC A61F 13/00068; A61M 1/0088; A61H 7/00
USPC 601/6; 604/319; 604/304

(57) ABSTRACT

Various embodiments of a wound cover apparatus are provided. In one embodiment a wound cover apparatus includes a treatment enclosure and a vacuum seal that is external to the treatment enclosure. The vacuum seal includes a vacuum passageway and a vacuum port in fluid communication with one another. In another embodiment the apparatus further includes a treatment port in fluid communication with the treatment enclosure. The wound cover apparatus adheres to the patient by application of vacuum applied to the treatment enclosure and the vacuum seal which extends from the housing. The negative pressure applied to the wound cover apparatus creates negative stress along periwound which promotes perfusion and vascularization and induces mechanical deformation to accelerate healing.

### RELATED APPLICATION

This application claims priority from U.S. Provisional Application No. 61/756,005, filed 24 Jan. 2013, the subject matter of which is incorporated herein by reference in its entirety.

### TECHNICAL FIELD

The present invention relates generally to an apparatus for medical treatment. More specifically, the disclosure relates to an apparatus that uses negative pressure to promote healing of wounds.

### BACKGROUND OF THE INVENTION

Several medical devices are used in conjunction with negative pressure for patient treatment. For example, the application of negative pressure, or vacuum, in medical devices has been used for the treatment of a variety of wounds. Typically, such devices that use vacuum are placed over a wound site to remove exudate generated by the wound. These devices are applied against the patient's body but they can cause localized injury to the skin or the wound and also to the underlying tissue. A pressure ulcer is an example of a localized injury to the skin, and in some cases the underlying tissue, that can result from direct pressure or shear and friction on the skin. This injury can commonly occur over a bony prominence, and a number of contributing or confounding factors are also associated with pressure ulcers. Although the application of negative pressure has been used to facilitate patient treatment, the use of some conventional devices does not promote healing and can present issues relating to discomfort, skin adhesion, and injury.

### SUMMARY OF THE INVENTION

The wound cover apparatus, according to an embodiment of the present invention, includes a treatment enclosure and a vacuum seal that is external to the treatment enclosure. The wound cover apparatus also includes a vacuum port in fluid communication with a vacuum passageway of the vacuum seal. In another embodiment the wound cover apparatus further includes a treatment port for application of positive or negative gas pressure to the treatment enclosure. The wound cover apparatus adheres to the patient and accelerates wound healing by application of vacuum applied to the treatment enclosure and the vacuum seal which extends outward from the treatment enclosure and is disposed along the periwound.

In another embodiment, a method of medical treatment includes placing a wound cover apparatus onto a user, such as a patient, and applying negative pressure to a vacuum seal that is external to a treatment enclosure. The negative pressure or vacuum creates suction along the vacuum seal to accelerate healing and prevent injury to the user.

### BRIEF DESCRIPTION OF THE DRAWINGS

The example embodiments of the present invention can be understood with reference to the following drawings. The components in the drawings are not necessarily to scale. Also, in the drawings, like reference numerals designate corresponding parts throughout the several views.

FIG. 1 is a top, external-view illustration of a wound cover apparatus, according to an embodiment of the present invention;

FIG. 2 is an exploded, internal-view illustration of the wound cover apparatus of FIG. 1, according to an embodiment of the present invention;

FIG. 3 is a cross-sectional view of the wound cover apparatus taken along lines 3-3 of FIG. 2, according to an embodiment of the present invention;

FIG. 4 is a cross-sectional view of the wound cover apparatus taken along lines 4-4 of FIG. 2, according to an embodiment of the present invention;

FIG. 5 is a cross-sectional view of an alternative wound cover apparatus, according to an embodiment of the present invention;

FIG. 6 is a cross-sectional view of an alternative wound cover apparatus, according to an embodiment of the present invention;

FIG. 7 is a perspective cut-away view of an alternative wound cover apparatus that further includes a seal interface, according to the embodiment of the present invention;

FIG. 8 is a break-out view of a portion of the wound cover apparatus of FIG. 7 that has been cut away from the treatment apparatus; and

FIG. 9 is an exploded, internal-view illustration of an alternative wound-cover treatment apparatus, according to an embodiment of the present invention.

### DETAILED DESCRIPTION

The various embodiments of an apparatus and methods for treating wounds and injuries by applying pressure against a body or skin are described herein. It should be understood that “vacuum” or “negative pressure” refers to reduced pressure relative to atmospheric pressure, or sub-atmospheric pressure.

As will be further described below, the application of vacuum not only secures the wound cover apparatus to the patient or subject but also accelerates healing. Vacuum is applied to the apparatus along the wound proper and beyond the wound, that is, the area between the wound and the free edge of the skin. The apparatus with applied vacuum promotes delivery of nutritive blood to the tissue to form new vessels, and also creates mechanical pulling of tissue along the wound edge to shrink the wound. In example embodiments, the vacuum can be continuous, intermittent and variable.

Wounds resulting from a variety of causes including, but not limited to, pressure, arterial inflow and/or venous outflow abnormalities, diabetic non-healing ulcers, skin tears, tape burns, perineal dermatitis, maceration or excoriation, for example, can be more effectively treated with the use of the embodiments of the wound cover apparatus described herein. In addition, the embodiments of the wound cover apparatus herein protect the skin surrounding the wound unlike conventional adhesives which are known to cause additional damage.

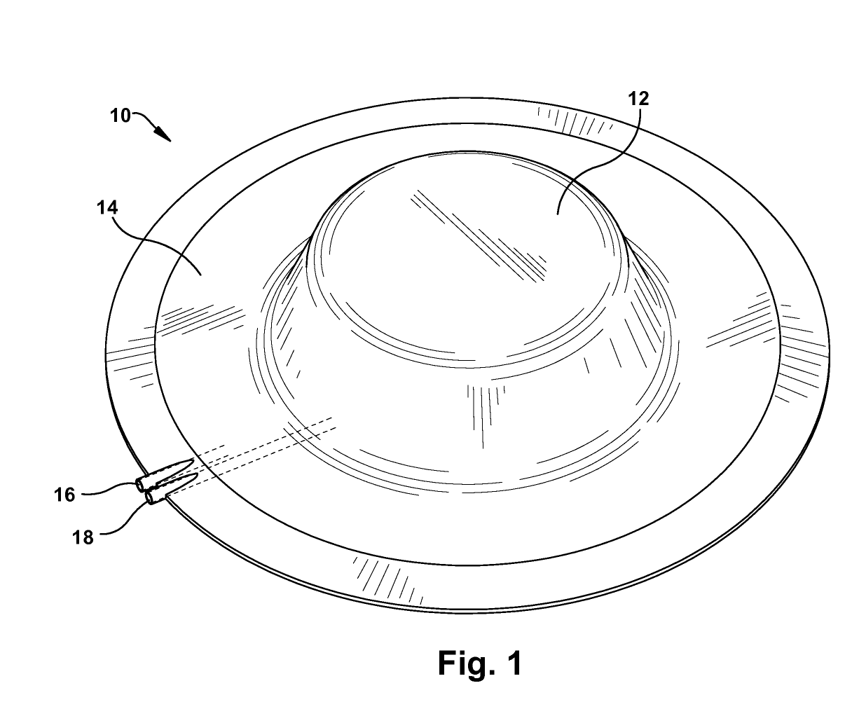

FIG. 1 is a top, external-view illustration of a wound cover apparatus 10, according to an embodiment of the present invention. The wound cover apparatus 10 is described herein as an apparatus applied to a wound site to improve healing, however, it should be understood that wound cover apparatus 10 may have other uses. Wound cover apparatus 10 includes a treatment enclosure 12 and a vacuum seal 14. As shown, the treatment enclosure 12 and vacuum seal 14 are an integral structure in which the vacuum seal 14 extends outward from the treatment enclosure, for example, as a single molded unit. Alternatively, the treatment enclosure 12 and vacuum seal 14 can also be separate components physically attached or connected together.

The vacuum-assist wound cover apparatus is positioned such that the treatment enclosure 12 is disposed above the wound of a patient or subject, and vacuum seal 14 which extends outward from the treatment enclosure and is disposed between the wound site and the free edge of skin, i.e. the periwound. The vacuum-assist wound cover apparatus 10 maintains an effective seal against the patient to hold the apparatus securely in place.

The body fluid created on a wound site can be a physical and chemical deterrent to healing. The vacuum under the treatment enclosure removes or drains excessive fluid and debris including bacteria. Apparatus 10 with applied vacuum inside treatment enclosure 12 and vacuum seal 14 creates “microstrain” which is a microdeformation at the cellular level which leads to cell stretch. Microstrain reduces edema and promotes perfusion via delivery of nutritive blood to the tissue to increase cell growth and division. The result is formation of granulation tissue and blood vessels, i.e. vascularization, to accelerate healing,

The vacuum applied to vacuum seal 14 outside treatment enclosure 12 creates an inward tension of skin or periwound cells that prevents the wound from increasing in size and also induces mechanical deformation of the wound edge tissue. “Macrostrain” is a visible stretch of the skin that draws wound edges together. Although not wishing to be bound by a particular theory, microstrain and the compression of the wound edge tissue may decrease the blood flow and stimulate granulation tissue formation at the wound edge to accelerate healing. The pressure applied to the area surrounding the wound can be more effectively treated with the use of the wound cover apparatus 10.

Vacuum seal 14 includes a vacuum port 16 which provides access for negative pressure connection. During use, the wound cover apparatus 10 can be placed onto the patient's body such that the wound cover is positioned directly above the wound to be treated. The negative pressure or vacuum suctions air out of the unit through vacuum port 16 so that the vacuum seal 14 adheres to the body of a patient. The apparatus can provide a substantially even distribution of pressure, although in some embodiments the pressure may vary along different regions of the apparatus. The vacuum or negative pressure may be applied continuously, intermittently and/or may be variable. Intermittent or variable pressure can stimulate biological mechanisms that promote healing.

In another embodiment the wound cover apparatus 10 further includes a treatment port 18 that is in fluid communication with wound cover 12. Treatment port 18, as shown in FIG. 1, extends from vacuum seal 14. Vacuum port 16 and treatment port 18 are shown side by side in a parallel arrangement for convenient hook up to auxiliary equipment, although the locations of each of the vacuum port 16 and treatment port 18 may vary. Treatment port 18 and extends beneath the surface of the vacuum seal and into treatment enclosure 12 (shown in phantom) and provides access for positive gas pressure, negative gas pressure, or delivery of a liquid, for example a therapeutic agent. Accordingly, in one example embodiment, a vacuum may be applied to remove wound exudates out of treatment enclosure 12. In another embodiment treatment port 18 is used to deliver therapeutic agents to the wound via positive pressure. The wound cover apparatus 10 in its entirety or individual portions of the apparatus, such as the vacuum seal 14 and the treatment enclosure 12, can be made of a flexible material to conform to the surface contours of the patient's body. Flexible materials include but are not limited to, thermoplastic polymers, thermoset polymers, fabric structures, etc., and are further described below. The various portions of the wound cover apparatus 10 can be the same material or different materials. For example, the vacuum seal 14 can be a material that is softer and more flexible than the material that forms the treatment enclosure 12, which can be relatively rigid to withstand pressure and prevent collapse. The wound cover apparatus can be placed flat against a patient's body or wrapped around curvatures for the treatment of a wound and to facilitate healing.

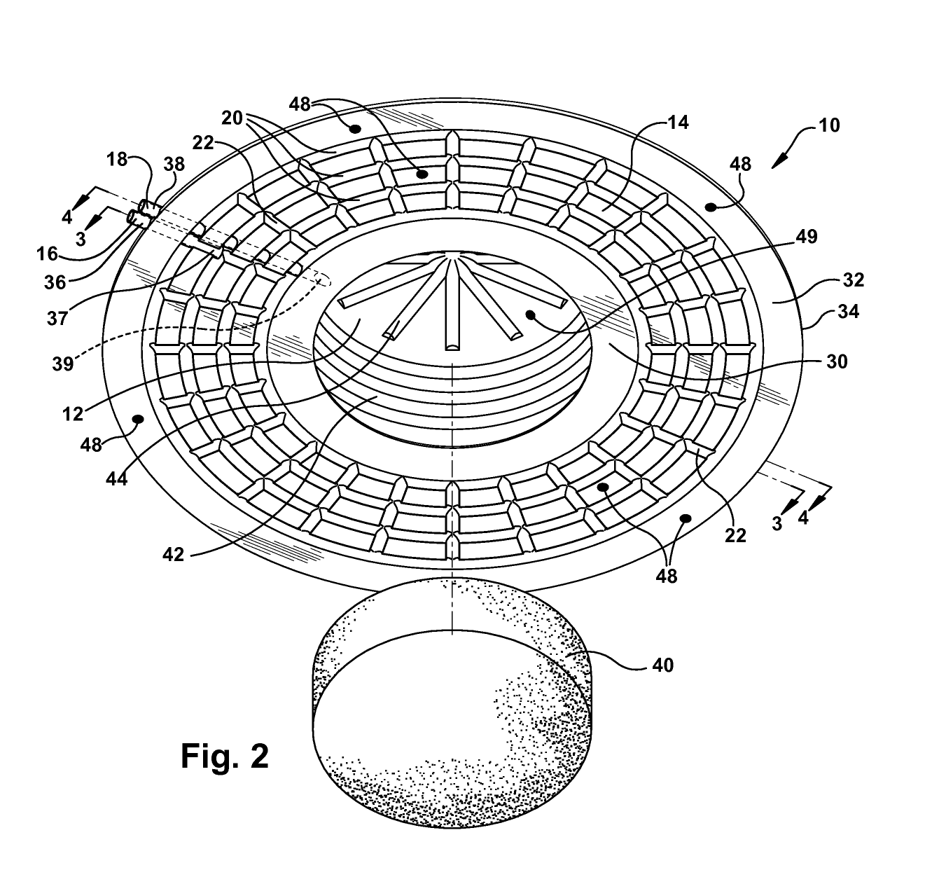

FIG. 2 is an exploded, internal-view illustration of the vacuum-assisted wound cover apparatus of FIG. 1. The underside view of vacuum seal 14 reveals a plurality of vacuum passageways 20 in communication with vacuum port 16. Vacuum passageways 20 are shown as open, concave channels that are substantially parallel to one another. In another embodiment a single vacuum passageway 20 may be sufficient. The number of vacuum passageways 20 present may depend on the size or surface area of vacuum seal 14. The pattern or location of passageways 20 may be selected to achieve an even distribution of pressure, for example, or to reduce or eliminate shear and friction forces directed to the wound.

As shown in the example embodiment of FIG. 2, the vacuum passageways 20 may include at least one intersecting channel such as, for example, transverse channel 22. Transverse channel 22 is optional but it increases the area of the vacuum passageway and provides a more equal force distribution along the vacuum seal 14. FIG. 2 illustrates a plurality of radial channels 22 that radiate outward toward edge 24 of the vacuum seal 14. Vacuum passageways 20 are also shown as a substantially circular pattern about the vacuum seal 14, i.e. a pattern of concentric circles, and conform to the general shape or perimeter of both treatment enclosure 12 and vacuum seal 14, however, a variety of patterns and shapes are also suitable.

The wound cover apparatus 10 can have a variety of overall shapes. Shapes other than the round shape of the apparatus 10, the treatment enclosure 12 and the vacuum seal 14 are possible. For example, a wound cover apparatus may have a rectangular treatment enclosure 12 and vacuum seal 14, and the vacuum passageways disposed therein may conform to a rectangular pattern.

Vacuum seal 14 can optionally include a valve portion 30 disposed between the vacuum passageways 20 and treatment enclosure 12. Valve portion 30 is substantially flat or smooth and serves as a border that prevents gas and/or fluids from seeping between treatment enclosure 12 and vacuum seal 14. Vacuum seal 14 also includes a flange portion 32 disposed along the outer periphery 34 of vacuum seal.

The vacuum seal 14, or portions thereof, for example the valve portion 30 or flange portion 32 or both, can optionally include a layer of adhesive for improved sealing to the skin. For example, adhesives that can adhere to patient's skin and easily removed without damage are recommended. As an example, a line of suitable adhesives include Silbione® silicone products available from Bluestar Silicones France SAS of Lyon, France.

In another embodiment wound cover apparatus can optionally include one or more pressure sensors 48 to monitor the vacuum pressure or force of adherence of the apparatus 10 to the patient while in use. The sensor 48 is capable of detecting the actual pressure which is compared to a preset threshold pressure that is based on the force needed to maintain the apparatus in position. The sensor 48 provides a signal to a vacuum pump controller (not shown) that can determine when more or less pressure is needed. For example, if the sensor 48 sends a signal that indicates that the actual vacuum is a weak or low vacuum, the vacuum pump controller determines whether it is below the pressure force threshold, and the pump provides more vacuum to the apparatus 10 so that it is more secure. If the sensor detects a high or strong vacuum which exerts a stronger pull force than is needed to retain the apparatus in position, then the sensor will signal the vacuum pump controller which will decrease the vacuum accordingly. The sensors can facilitate improved treatment by increasing comfort to the patient and promoting healing, and reducing damage.

Still referring to FIG. 2 the underside view of wound cover apparatus 10 and vacuum seal 14 reveals the construction of vacuum port 16 and treatment port 18, according to an example embodiment of the present invention. Vacuum port 16 is formed by a tube, for example, that has a proximal end 36 that extends beyond the peripheral edge 34 of vacuum seal 14 and a distal end 37 that extends to the vacuum passageway 20 and is in fluid communication with vacuum passageway 20 and interconnecting transverse channel 22. Treatment port 18 is formed by a tube that has a proximal end 38 that extends beyond the peripheral edge 34 of vacuum seal 14 and a distal end 39 (shown in phantom) that extends into the treatment enclosure 12 and is shown adjacent to valve 30.

In another embodiment, including the embodiments described above, wound cover apparatus 10 further includes a wound dressing 40. Wound dressing 40 can comprise several materials, including but not limited to, a sponge, fabric such as gauze, coated fabric, etc. The wound dressing can be made of several materials that may be coated or otherwise include coated layers for elution of therapeutic agents into the wound or skin. Wound dressing 40 may receive therapeutic agents, via treatment port 18 to aid in treatment of the wound and to reduce the risk of infection. Therapeutic agents include for example, pharmacological, biological components, and other components such as components with a programmable release pharmacokinetics. A wide variety of therapeutic agents can be used and will be discussed in more detail below.

As described above, the treatment enclosure 12 can be shaped in a variety of geometric shapes and sizes to treat a variety of wounds and accommodate a variety of wound dressings 40 shapes and sizes. Treatment enclosure 12 can include one or more ridges 42 to assist in retaining the wound dressing 40 into position. Treatment enclosure 12 also includes grooves 44 along the inside surface, for example the top surface, to drain and remove exudate fluids. Grooves 44 can be molded into the treatment enclosure 12 during injection molding or another manufacturing method.

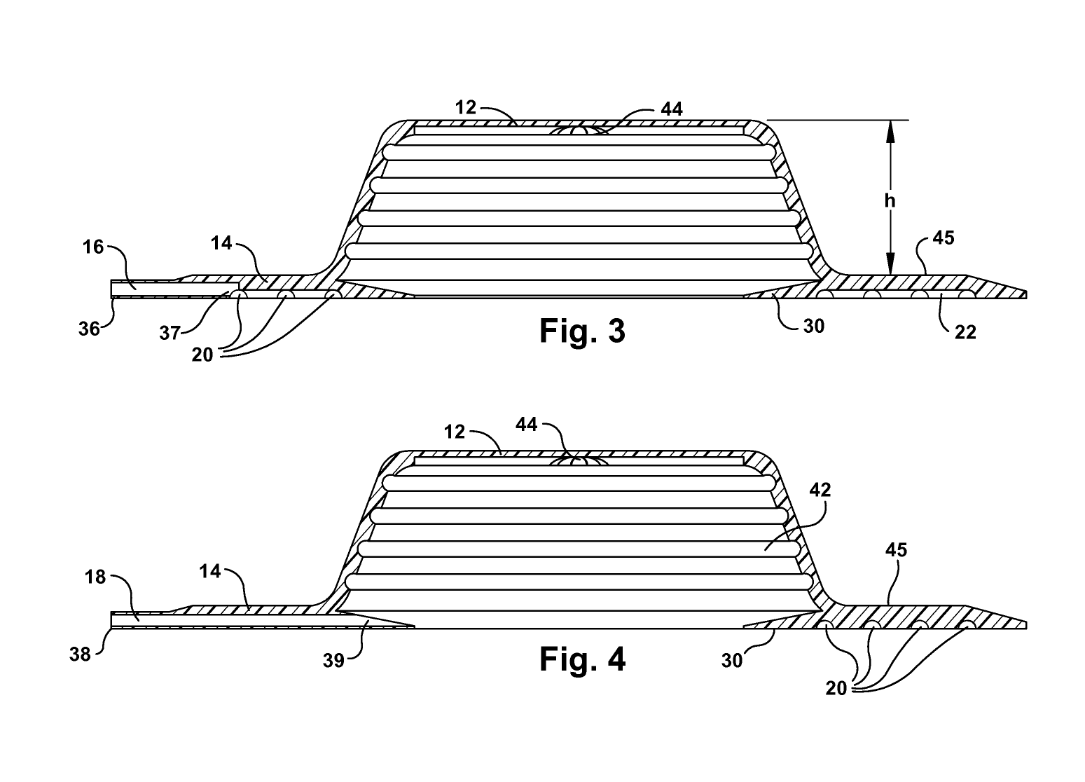

FIG. 3 is a cross-sectional view of the wound cover apparatus 10 taken along lines 3-3 of FIG. 2. The cross-section cuts along the length of the vacuum port 16 on one side of treatment enclosure 12 and along transverse passageway 22 on the opposite side of treatment enclosure. The proximal end 36 of vacuum port 16 extends beyond the perimeter of vacuum seal 14 and the distal end 37 joins vacuum passageway 20 so that vacuum port 16 is in fluid communication with the plurality of passageways 20 and transverse passageways 22 in the vacuum seal 14. Valve 30 extends inside treatment enclosure 12 and its top surface 47 is optionally tapered, as shown, so that the fluids generated by the wound remain in the treatment enclosure 12.

The height, h, of treatment enclosure 12, as well as the shape of treatment enclosure 12, can vary to fit the wound dressing 40 (FIG. 1) and can be flush with the top surface 45 of vacuum seal 14. Therefore the height, h, can be greater or equal to zero, and typically, the height ranges from greater than zero to about 100 centimeters to accommodate many different sizes of wound dressings. The treatment enclosure 12 has a thicker wall section at the center with the presence of grooves 44. As mentioned above, the shape of the wound cover 10 can vary and conform to the general shape or perimeter of the wound to be treated as well as both treatment enclosure 12 and vacuum seal 14. Other patterns and shapes are possible.

FIG. 4 is a cross-sectional view of the wound cover apparatus taken along lines 4-4 of FIG. 2. The cross-section is taken through treatment port 18 on one end and across vacuum seal passageways 20. The proximal end 38 of treatment port 16 extends beyond the perimeter of vacuum seal 14 and the distal end 39 extends into the treatment enclosure 12. The distal end 39 of treatment port 16 is optionally tapered, as shown, so that the opening is larger to pull additional fluid out of the treatment enclosure and to prevent fluid from travelling under valve 30 and into vacuum passageways 20.

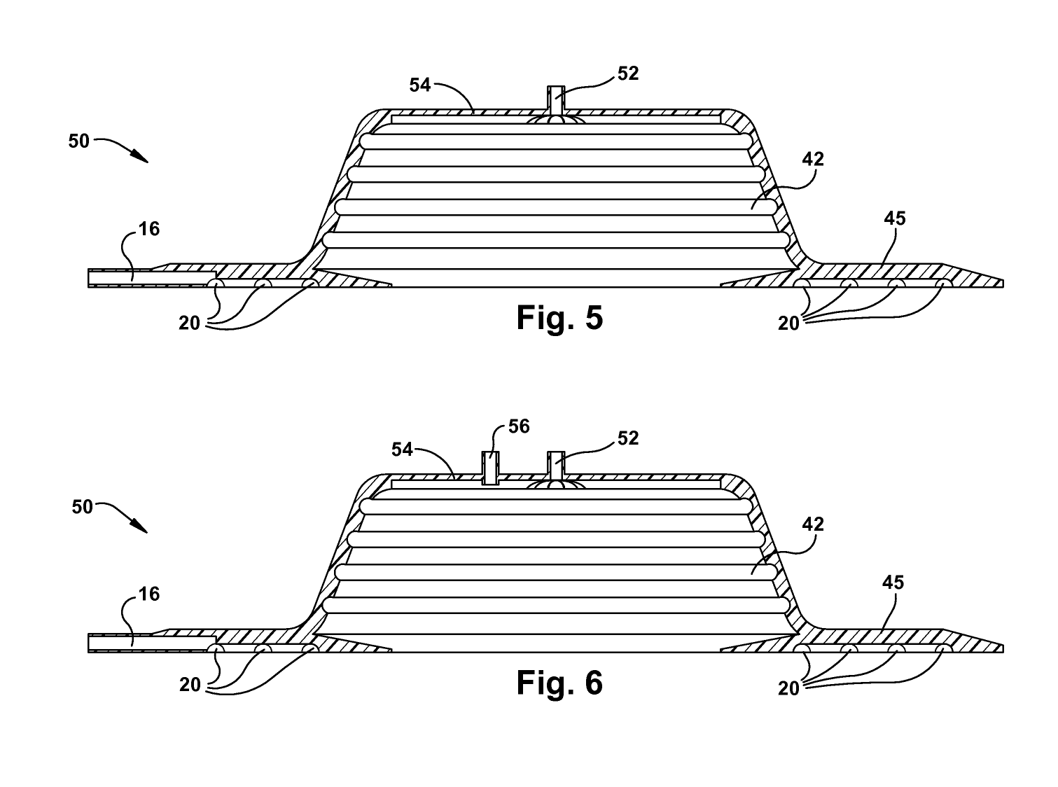

FIG. 5 is a cross-sectional view of an alternative wound cover apparatus 50, according to an embodiment of the present invention. Wound cover apparatus 50 is similar to wound cover apparatus 10 of the embodiments shown in FIGS. 1 through 4, and further includes vacuum port 52 located along top surface of treatment enclosure 54. Vacuum port 52 can be located along one of many alternative locations along treatment enclosure 54. Vacuum port 52 can be used to suction wound exudates, infectious materials, or other liquids. For example, the wound may be irrigated with water or a therapeutic agent followed by suctioning through vacuum port 52.

In another embodiment, FIG. 6 illustrates a cross-sectional view of the wound-cover treatment apparatus also taken along lines 3-3 of FIG. 2. Wound cover apparatus 50 is similar to wound cover apparatus 50 (FIG. 5) having vacuum port 16, and further includes vacuum port treatment port 56 located along surface of treatment enclosure 54. Treatment port 56 may be in lieu of a treatment port which extends from vacuum seal 14 such as treatment port 18 (FIG. 4), and alternatively, treatment port 56 may be a second treatment in addition to treatment port 18 (FIG. 4).

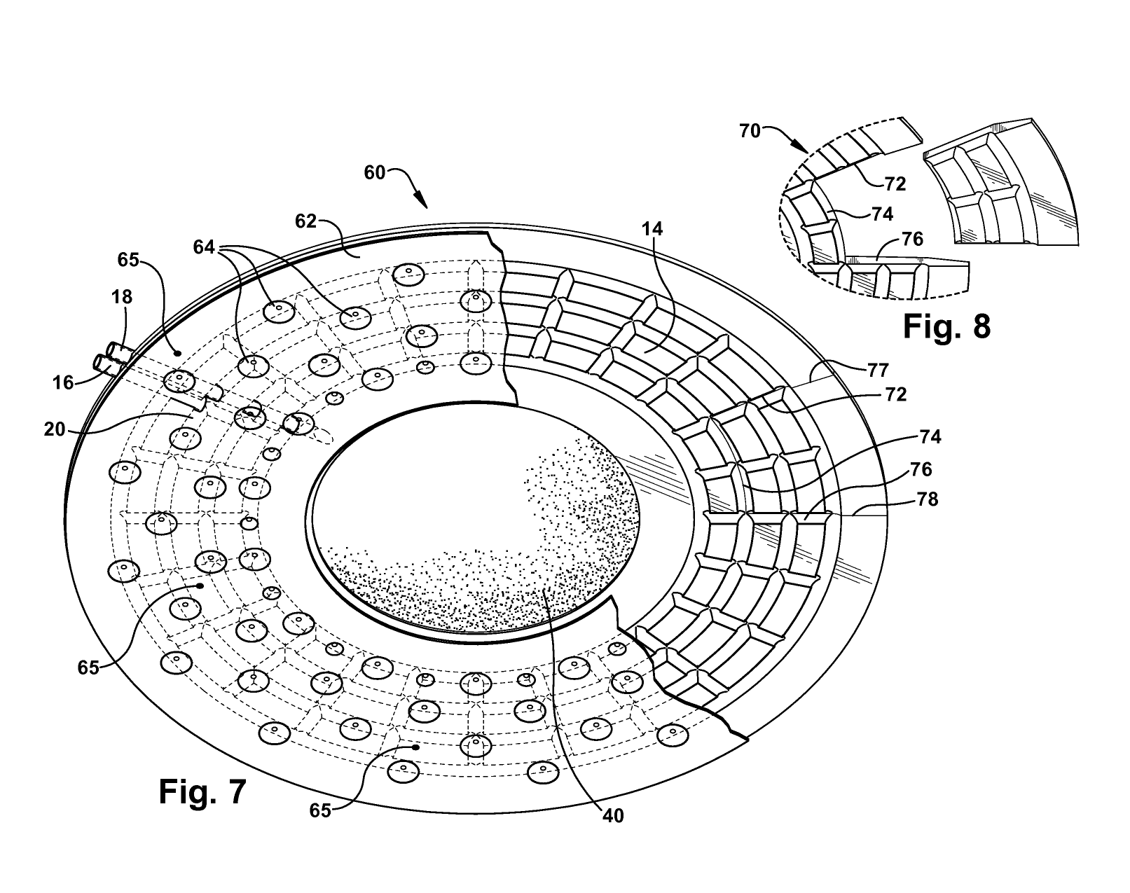

FIG. 7 is a perspective cut-away view of an alternative wound-cover treatment apparatus 60, according to an embodiment of the present invention. In any of the embodiments described above, the wound cover apparatus can further include a seal interface 62. Seal interface 62 is joined to the inside surface of vacuum seal 14, around the wound dressing 40, and becomes the layer that is in contact with the patient during use. Seal interface 62 comprises a plurality of openings 64 which are in communication with the vacuum passageway 20 and vacuum port 16. Openings 64 are shown as circular or round openings but can have a variety of alternative shapes, for example, oval, rectangular, polygonal, etc. In operation, vacuum is applied to the treatment apparatus 60 and creates a suction that is substantially uniform along the seal interface 62 to provide improved sealing of the apparatus. Similar to the sensors 48 of vacuum seal 14 described above (FIG. 2), seal interface 62 can optionally include sensors 65 to detect the vacuum pressure at the surface where the wound cover apparatus 60 contacts the patient.

Seal interface 62 can be secured to vacuum seal 14 by a variety of known materials or methods, for example, adhesives, heat sealing, etc., or the seal can be secured via a force fit of tight dimensional tolerances. The seal interface 62 and vacuum seal 14 can be manufactured as one integrated component, for example, as a single injection-molded unit. Seal interface 62 can optionally include a layer of adhesive for improved sealing, as described above in reference to portions or all of vacuum seal 14 described above. Likewise, seal interface 62 can optionally include a therapeutic agent for elution into the body or skin.

In any of the various embodiments described above, the wound-cover treatment apparatus is designed to facilitate multiple shape and size configurations and can be easily cut with an instrument, for example scissors, to improve the fit of the apparatus onto a particular body location e.g. arm, leg, chest, etc., being treated. The apparatus may be symmetrical or asymmetrical. FIG. 8 is a break-out view illustration showing that a portion 70 of vacuum seal 14 of FIG. 7 has been cut along lines 77 and 78 and vent channels 72, 74 and 76. In embodiments where the wound cover apparatus includes seal interface 62, the seal interface is also cut with the portion of vacuum seal 14 that is removed.

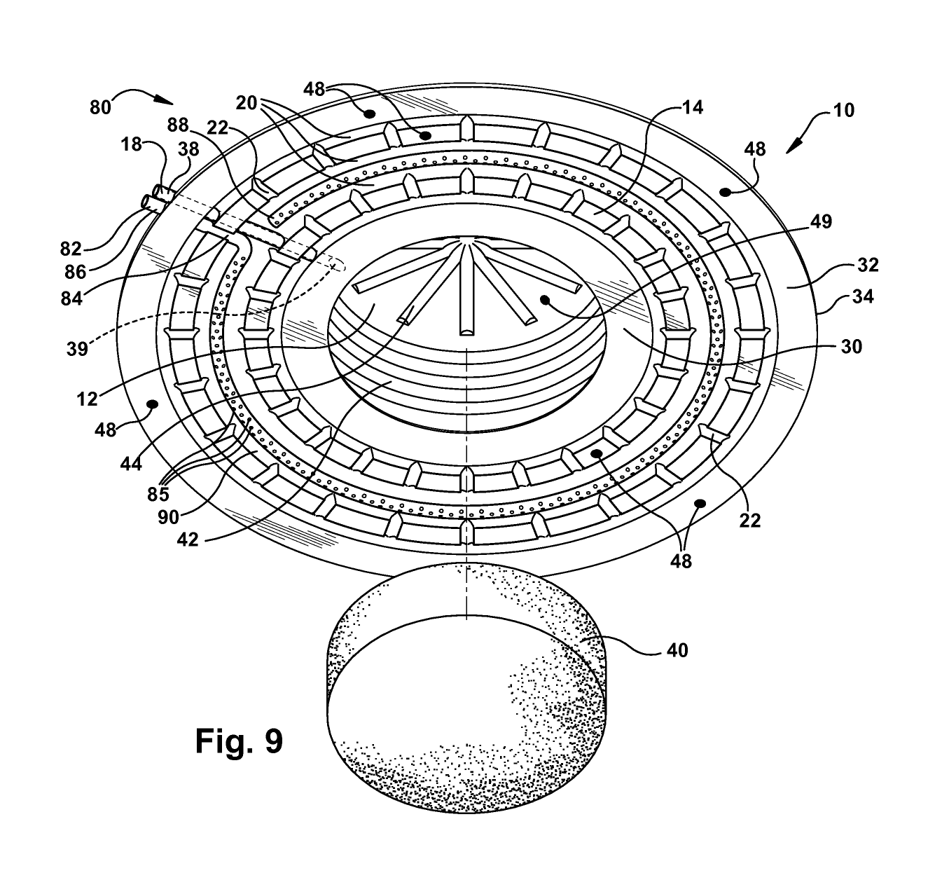

FIG. 9 illustrates an exploded, internal-view illustration of an alternative wound-cover treatment apparatus 80, according to another embodiment of the present invention. Wound-cover treatment apparatus 80 shown in FIG. 9 includes many of the features of FIG. 2 and includes an alternative vacuum port 82. Vacuum port 82 is an interface that links the vacuum source with the vacuum passageways of the vacuum seal 14. Vacuum port 82 is a perforated vacuum tube 84 having a plurality of openings 85 for vacuum distribution that is substantially uniform along the vacuum seal 14. In another embodiment vacuum port 82 may include two or more perforated vacuum tubes 84. Perforated tube 84 has a proximal end 86 that extends beyond the peripheral edge 34 of vacuum seal 14 and a distal end 88. The perforated tubing 84 extends along passageway 90 of vacuum seal 14 and is shown as a ring shape, however, the perforated tubing can have a different length and configured in a different shape. For example, the perforated tubing can have a shorter length and be shaped as a horseshoe or U-shaped, a semi-circle, etc. The vacuum seal 14 can also have an alternative shape, as described above, and the perforated tubing may or may not conform to the shape of the vacuum seal. The features in any of the embodiments described above and shown in FIGS. 1-8 can include vacuum port.

In any of the embodiments described herein, wound cover apparatus 10, or portions thereof such as the vacuum seal 14 and seal interface 62 can be the same material or different materials. Also, vacuum seal 14 and seal interface 62 can each be a double structure that includes a first material composition plus an additional layer of a second material composition. The materials used can be selected or modified so for easy processing, for example, via injection molding, blow molding, thermoforming, etc. Suitable materials include, but are not limited to, thermoset and thermoplastic polymers and blends thereof. Examples of such polymers include, but are not limited to, silicones, polyamide, polyester, polycarbonate, polyetherimide, latex, polyurethane, polyolefin, polyvinyl chloride (PVC), ethylene vinyl acetate (EVA), ethylene methylacrylate (EMA), ethylene ethylacrylate (EEA), styrene butadiene styrene (SBS), and ethylene propylene diene rubber (EPDM), and blends thereof. The material composition of vacuum seal 14 and seal interface 62 are selected depending upon the desired surface characteristics, chemical compatibility, and other design criterion of the wound-cover treatment apparatus. For example, seal interface 62 and vacuum seal 14 if seal interface 62 is not present, should not grab the skin or exert tangential stresses on the skin directly or indirectly, as such lateral stresses transmitted to the skin are likely to cause damage.

As mentioned above, the various embodiments of the wound cover apparatus 10, 50, 60, 80, or portions thereof, for example the vacuum seal 14, seal interface 62 and/or the wound dressing 40, can be treated or embedded with at least one therapeutic agent for elution into the body to accelerate healing or to prevent infection. The therapeutic agent is capable of preventing a variety of pathological conditions including, but not limited to, arrhythmias, thrombosis, stenosis, apoptosis, and inflammation. Accordingly, the therapeutic agent may include at least one of the following: an anti-arrhythmic agent; anticoagulant; an antioxidant; a fibrinolytic; a steroid; an anti-apoptotic agent; an anti-overgrowth agent (i.e., capable of preventing epithelial cell overgrowth); and/or an anti-inflammatory agent. Optionally or additionally, the therapeutic agent may be capable of treating or preventing other disease or disease processes such as microbial infections and heart failure. In these instances, the therapeutic agent may include an anti-microbial agent, an inotropic agent, a chronotropic agent, and/or a biological agent such as a cell or protein. More specific types of these therapeutic agents are listed below.

Examples of acceptable therapeutic agents include heparin, synthetic heparin analogues (e.g., fondaparinux), G(GP) II_{b}/IIIₐ inhibitors, vitronectin receptor antagonists, hirudin, antithrombin III, drotrecogin alpha; fibrinolytics such as alteplase, plasmin, lysokinase, factor XIIa, factor VIIa, prourokinase, urokinase, streptokinase; thrombocyte aggregation inhibitors such as ticlopidine, clopidogrel, abciximab, dextrans; corticosteroids such as aldlometasones, estradiols, such as 17β-estradiol, amcinonides, augmented betamethasones, beclomethasones, betamethasones, budesonides, cortisones, clobetasol, clocortolones, desonides, desoximetasones, dexamethasones, flucinolones, fluocinonides, flurandrenolides, flunisolides, fluticasones, halcinonides, halobetasol, hydrocortisones, methylprednisolones, mometasones, prednicarbates, prednisones, prednisolones, triamcinolones; fibrinolytic agents such as tissue plasminogen activator, streptokinase, dipyridamole, ticlopidine, clopidine, and abciximab; non-steroidal anti-inflammatory drugs such as salicyclic acid and salicyclic acid derivatives, para-aminophenol derivatives, indole and indene acetic acids (e.g., etodolac, indomethacin, and sulindac), heteroaryl acetic acids (e.g., ketorolac, diclofenac, and tolmetin), arylpropionic acids (e.g., ibuprofen and derivatives thereof), anthranilic acids (e.g., meclofenamates and mefenamic acid), enolic acids (e.g., piroxicam, tenoxicam, phenylbutazone, and oxyphenthatrazone), gold compounds (e.g., auranofin, aurothioglucose, and gold sodium thiomalate), diflunisal, meloxicam, nabumetones, naproxen, oxaprozin, salsalate, celecoxib, rofecoxib; cytostatics such as alkaloids and podophyllum toxins such as vinblastin, vincristin; alkylants such as nitrosoureas and nitrogen lost analogues; cytotoxic antibiotics such as daunorubicin, doxorubicin, and other anthracyclins and related substances, bleomycin, and mitomycin; antimetabolites such as folic acid analogues, purine analogues and related inhibitors (e.g., mercaptopurine, thioguanine, pentostatin, and 2-chlorodeoxyadenosine), pyrimidine analogues (e.g., fluorouracil, floxuridine, and cytarabine), and platinum coordination complexes (e.g., cisplatinum, carboplatinum and oxaliplatinum); tacrolimus, azathioprine, cyclosporine, paclitaxel, docetaxel, sirolimus; amsacrin, irinotecan, imatinib, topotecan, interferon-alpha 2a, interferon-alpha 2b, hydroxycarbamide, miltefosin, pentostatin, porfimer, aldesleukin, bexarotene, and tretinoin; antiandrogens and antiestrogens; antiarrythmics, in particular antiarrhythmics of class I such as antiarrhythmics of the quinidine type (e.g., quinidine, dysopyramide, ajmaline, prajmalium bitartrate, and detajmium bitartrate); antiarrhythmics of the lidocaine type, (e.g., lidocaine, mexiletin, phenyloin, and tocainid); antiarrhythmics of class I C (e.g., propafenone, flecainide (acetate)); antiarrhythmics of class II, including betareceptor blockers such as metoprolol, esmolol, propranolol, metoprolol, atenolol, and oxprenolol; antiarrhythmics of class III such as amiodarone and sotalol; antiarrhythmics of class IV such as diltiazem, and verapamil; and other antiarrhythmics such as adenosine, orciprenaline, TC-912, endothelin antagonists, phosphodiesterase-5 (PDE-5) inhibitors, prostaglandins (e.g., thromboxane, prostacyclin, and prostaglandin D, E and F), ipratropium bromide, and novel anti-proliferative agents, such as imatinib (GLEEVEC).

Other types of therapeutic agents may include digitalis glycosides such as acetyl digoxin/methyldigoxin, digitoxin, and digoxin; heart glycosides such as ouabain and proscillaridin; antihypertensives such as centrally effective antiadrenergic substances (e.g., methyldopa and imidazoline receptor agonists); calcium channel blockers of the dihydropyridine type, such as nifedipine and nitrendipine; ACE inhibitors (e.g., quinaprilate, cilazapril, moexipril, trandolapril, spirapril, imidapril, and trandolapril); angiotensin-II-antagonists (e.g., candesartancilexetil, valsartan, telmisartan, olmesartan medoxomil, and eprosartan); peripherally effective alpha-receptor blockers such as prazosin, urapidil, doxazosin, bunazosin, terazosin, and indoramin; vasodilators such as dihydralazine, diisopropyl amine dichloroacetate, minoxidil, and nitropiusside-sodium; other antihypertonics such as indapamide, codergocrin mesilate, dihydroergotoxin methane sulphonate, cicletanin, bosentan, and fluocortisone; phosphodiesterase inhibitors, such as milrinone and enoximone, as well as antihypotonics (e.g., adrenergics and dopaminergic substances such as dobutamine, epinephrine, etilefrine, norfenefrine, norepinephrine, oxilofrine, dopamine, midodrine, pholedrine, and amezinium methyl) and partial adrenoreceptor agonists (e.g., dihydroergotamine); fibronectin, polylysines and ethylene vinyl acetates; and adhesive substances such as cyanoacrylates, beryllium, and silica.

Additional therapeutic agents may also include antibiotics and anti-infectives, such as: β-lactam antibiotics (e.g., β-lactamase-sensitive penicillins, including benzyl penicillins (penicillin G) and phenoxymethylpenicillin (penicillin V)); β-lactamase-resistant penicillins, such as aminopenicillins, which include amoxicillin, ampicillin, and bacampicillin; acylaminopenicillins such as mezlocillin and piperacillin; carboxypenicillines and cephalosporins (e.g., cefazolin, cefuroxim, cefoxitin, cefotiam, cefaclor, cefadroxil, cefalexin, loracarbef, cefixime, cefuroximaxetil, ceftibuten, cefpodoximproxetil, and cefpodoximproxetil); aztreonam, ertapenem, and meropenem; β-lactamase inhibitors such as sulbactam and sultamicillintosilates; tetracyclines such as doxycycline, minocycline, tetracycline, chlorotetracycline, oxytetracycline; aminoglycosides such as gentamicin, neomycin, streptomycin, tobramycin, amikasin, netilmicin, paromomycin, framycetin, and spectinomycin; makrolide antibiotics such as azithromycin, clarithromycin, erythromycin, roxithromycin, spiramycin, and josamycin; lincosamides such as clindamycin and lincomycin; gyrase inhibitors, such as fluoroquinolones, which include ciprofloxacin, ofloxacin, moxifloxacin, norfloxacin, gatifloxacin, enoxacin, fleroxacin, and levofloxacin; quinolones such as pipemidic acid; sulphonamides such as trimethoprim, sulphadiazin, and sulphalene; glycopeptide antibiotics such as vancomycin and teicoplanin; polypeptide antibiotics, such as polymyxins, which include colistin, polymyxin-b, and nitroimidazol derivatives (e.g., metronidazol and tinidazol); aminoquinolones such as chloroquin, mefloquin, and hydroxychloroquin; biguanides such as proguanil; quinine alkaloids and diaminopyrimidines such as pyrimethamine; amphenicols such as chloramphenicol; rifabutin, dapsone, fusidinic acid, fosfomycin, nifuratel, telithromycin, fusafungin, fosfomycin, pentamidindiisethionate, rifampicin, taurolidine, atovaquone, and linezolid; virostatics such as aciclovir, ganciclovir, famciclovir, foscamet, inosine (dimepranol-4-acetamidobenzoate), valganciclovir, valaciclovir, cidofovir, and brivudin; tyrosine kinase inhibitors; anti-apoptotic agents such as caspase inhibitors (e.g., fluoromethylketone peptide derivatives), calpain inhibitors, cathepsin inhibitors, nitric oxide synthase inhibitors, flavonoids, vitamin A, vitamin C, vitamin E, vitamin D, pycnogenol, super oxidedismutase, N-acetyl cysteine, selenium, catechins, alpha lipoic acid, melatonin, glutathione, zinc chelators, calcium chelators, and L-arginine; Coumadin; beta-blockers; diuretics; spirolactone; TC-313; and natural products such as vinca alkaloids (e.g., vinblastine, vincristine and vinorelbine).

As noted above, the therapeutic agent may also include a biological agent. The biological agent may include organic substances such as peptides, proteins, enzymes, carbohydrates (e.g., monosaccharides, oligosaccharides and polysacchardies), lipids, phospholipids, steroids, lipoproteins, glycoproteins, glycolipids, proteoglycans, polynucleotides (e.g., DNA and RNA), antisense polynucleotides (e.g., c-myc antisense), antibodies (e.g., monoclonal or polycolonal) and/or antibody fragments (e.g., anti-CD34 antibody), bioabsorbable polymers (e.g., polylactonic acid), chitosan, extracellular matrix modulators, such as matrix metalloproteinases (MMP), which include MMP-2, MMP-9 and Batimastat; and protease inhibitors.

Biological agents may include, for example, agents capable of stimulating angiogenesis in the myocardium. Such agents may include vascular endothelial growth factor (VEGF), basic fibroblast growth factor (bFGF), non-viral DNA, viral DNA, and endothelial growth factors (e.g., FGF-1, FGF-2, VEGF, TGF). Other growth factors may include erythropoietin and/or various hormones such as corticotropins, gonadotropins, thyrotrophin, desmopressin, terlipressin, oxytocin, cetrorelix, corticorelin, leuprorelin, triptorelin, gonadorelin, ganirelix, buserelin, nafarelin, and goserelin. Additional growth factors may also include cytokines, epidermal growth factors (EGF), platelet derived growth factor (PDGF), transforming growth factors-β (TGF-β), transforming growth factor-α (TGF-α), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumour necrosis factor-α (TNF-α), tumour necrosis factor-β (TNF-β), interferon-γ (INF-γ), colony stimulating factors (CSFs); monocyte chemotactic protein, and fibroblast stimulating factor 1.

Still other biological agents may include regulatory peptides such as somatostatin and octreotide; bisphosphonates (e.g., risedronates, pamidronates, ibandronates, zoledronic acid, clodronic acid, etidronic acid, alendronic acid, and tiludronic acid); fluorides such as disodium fluorophosphate and sodium fluoride; calcitonin and dihydrotachystyrene; histamine; fibrin or fibrinogen; endothelin-1; angiotensin II; collagens; bromocriptin; methylsergide; methotrexate; carbontetrachloride and thioacetamide.

The wound cover apparatus 10, or portions thereof, may also be treated (i.e., seeded) with other biological agents, such as cells. Suitable cells may include any one or combination of eukaryotic cells. Additionally or optionally, the cells may be capable of producing therapeutic agents and/or genetically engineered to produce therapeutic agents. Suitable cells for use in the present invention include, for example, progenitor cells such as stem cells. The cells may be autologous or allogenic, genetically engineered or non-engineered, and may include, for example, mesenchymal or mesodermal cells, including, but not limited to, endothelial progenitor cells, endothelial cells, and fibroblasts. Mixtures of such cells can also be used.

A variety of ex vivo or in vivo methods can be used to deliver a nucleic acid molecule or molecules, such as a gene or genes, to the cells. For example, the cells can be modified (i.e., genetically engineered) to produce or secrete any one or combination of the above therapeutic agents, including, but not limited to, anticoagulant agents, antiplatelet agents, antifibrinolytic agents, angiogenesis factors, and the like. Ex vivo gene transfer is a process by which cells are removed from the body using well known techniques, genetically manipulated, usually through transduction or transfection of a nucleic acid molecule into the cells in vitro, and then returned to the body for therapeutic purposes. This contrasts with in vivo genetic engineering where a gene transfer vector or a liposome that contains specific genes is administered to a patient resulting in genetic transfer into cells and tissues in the intact patient. Ex vivo and in vivo gene transfer techniques are well known to one of skill in the art.

To treat the wound cover apparatus, or components therof, with at least one therapeutic agent, a variety of methods, agents, and compositions may be used. For example, the therapeutic agent can be simply linked to the surface of the expandable support member 12, embedded and released from within polymer materials, such as a polymer matrix, or surrounded by and released through a carrier. Several approaches to treating medical devices with therapeutic agents exist. Some therapeutic agents can be loaded directly onto metallic surfaces; however, a coating composition, typically comprised of at least one polymer and at least one therapeutic agent, is usually used to treat drug-eluting devices. The coating composition ensures retention of the therapeutic agent during deployment and modulates elution kinetics of the therapeutic agent. By altering the release kinetics of different therapeutic agents in the same coating composition, distinct phases of a given disease process may be targeted.

As noted above, surfaces of the wound cover apparatus, or portions thereof including the vacuum seal 14, the seal interface 62 and the wound dressing 40, can be treated with a coating composition comprising at least one therapeutic agent and at least one dendrimer, polymer or oligomer material. The dendrimer(s), polymer(s) and/or oligomer(s) may be of various types and from various sources, including natural or synthetic polymers, which are biocompatible, bioabsorbable and useful for controlled release of the therapeutic agent. For example, synthetic polymers can include polyesters, such as polylactic acid, polyglycolic acid, and/or combinations thereof, polyanhydrides, polycaprolactones, polyhydroxybutyrate valerates, and other bioabsorbable polymers or mixtures of copolymers thereof. Natural polymeric materials can include proteins such as collagen, fibrin, elastin, extracellular matrix components, other biologic agents, and/or mixtures thereof.

The polymer material or mixtures thereof of the coating composition can include a therapeutic agent on the surface, and via a single layer. Optionally, multiple layers of the polymer material can be applied to form the coating composition. Multiple layers of the polymer material can also be applied between layers of the therapeutic agent. For example, the polymeric layers may be applied sequentially, with the first layer directly in contact with the uncoated surface of the apparatus and a second layer comprising the therapeutic agent and having one surface in contact with the first layer and the opposite surface in contact with a third layer of polymeric material which is in contact with the surrounding tissue. Additional layers of the polymeric material and therapeutic agent can be added as required.

Alternatively, the coating composition can be applied as multiple layers comprising one or more therapeutic agents surrounded by polymer material. For instance, the coating composition can comprise multiple layers of a single therapeutic agent, one or more therapeutic agents in each layer, and/or differing therapeutic agents in alternating layers. Alternatively, the layers comprising the therapeutic agent can be separated from one another by a layer of polymer material.

The coating composition may further comprise at least one pharmaceutically acceptable polymers and/or pharmaceutically acceptable carriers, for example, non-absorbable polymers, such as ethylene vinyl acetate and methylmethacrylate. The non-absorbable polymer, for example, can aid in further controlling release of the therapeutic agent by increasing the molecular weight of the coating composition and thereby delaying or slowing the rate of release of the therapeutic agent.

The coating composition can be applied to the present invention using standard techniques to cover the entire surface of the apparatus 10, or partially, as a single layer in a dot matrix pattern, for example. The coating composition can be applied using various techniques available in the art, such as dipping, spraying, vapor deposition, an injection-like and/or a dot matrix-like approach. Upon contact of the coating composition with adjacent tissue where implanted, the coating composition can begin to degrade in a controlled manner. As the coating composition degrades, the therapeutic agent is slowly released into adjacent tissue and/or the blood stream, and the therapeutic agent eluted so that the therapeutic agent can have its effect locally and/or downstream.

Where the therapeutic agent comprises a biological agent, such as cells, the biological agent can be coated directly onto the surface of the present invention or, alternatively, they can be incorporated into the polymeric material (e.g., into a polymer matrix). Such biological agents may also be included within at least one microscopic containment vehicle (e.g., a liposome, nanocapsule, nanoparticle, micelle, synthetic phospholipid, gas-dispersion, emulsion, microemulsion, nanosphere, and the like) that can be stimulated to release the biological agent(s) and/or that release the biological agent(s) in a controlled manner. The microscopic containment vehicle can be coated onto the surface of the present invention or incorporated into the polymeric material. Where the biological agent comprises cells, for example, the cells can be induced to produce, activate, and/or release their cellular products (including one or more therapeutic agents) by an external stimulation device (e.g., an electrical impulse). Alternatively, cells can constitutively release one or more therapeutic agents at a desired level.

Surfaces of wound cover apparatus 10, the wound dressing 40, or both, may further include a layer of biocompatible material. The layer of biocompatible material may be a synthetic material such as DACRON (Invista, Witchita, Kans.), GORE-TEX (W. L. Gore & Associates, Flagstaff, Ariz.), woven velour, polyurethane, polytetrafluoroethylene (PTFE), expanded PTFE (ePTFE), or heparin-coated fabric. Alternatively, the layer may be a biological material such as bovine or equine pericardium, peritoneal tissue, an allograft, a homograft, patient graft, or a cell-seeded tissue.

In any of the above-described embodiments, wound cover apparatus 10, 50, 60, 80 may include additional auxiliary equipment. As mentioned above the apparatus may also include a positive pressure source, a vacuum source, device that administers therapeutic materials, a disposable wound fluid collection container, for example. The flexible nature of the wound cover apparatus accommodates movement caused by pressure source, for example, intermittent operation of a compressor or vacuum pump, as well as movement by the user or contours of the patient body as discussed above. Wound cover apparatus can maintain a seal against the patient while reducing the stress of the surrounding skin or otherwise reducing the risk of injury.

Accordingly, a method of medical treatment, according to an embodiment of the present invention, includes placing the wound cover apparatus onto a patient and applying negative pressure through the vacuum passageway of a vacuum seal where the vacuum seal is external to a treatment enclosure of the apparatus. In another embodiment the method further includes applying positive pressure or applying negative pressure through treatment port of treatment enclosure while applying a negative pressure through the vacuum seal.

Although the invention has been described with reference to several specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

Having described the invention, we claim:

1. A wound cover apparatus comprising:
a treatment enclosure;
a vacuum seal external to the treatment enclosure comprising a vacuum passageway; and
a vacuum port in fluid communication with the vacuum passageway of the vacuum seal.

2. The wound cover apparatus of claim 1, comprising a treatment port in fluid communication with the treatment enclosure.

3. The wound cover apparatus of claim 1, comprising a vacuum port in fluid communication with the treatment enclosure.

4. The wound cover apparatus of claim 1, comprising:
a treatment port in fluid communication with the treatment enclosure; and
a vacuum port in fluid communication with the treatment enclosure.

5. The wound cover apparatus of claim 1, comprising at least two treatment ports in fluid communication with the treatment enclosure and a vacuum port in fluid communication with the treatment enclosure.

6. The wound cover apparatus of claim 1, wherein the vacuum seal surrounds the treatment enclosure in at least two dimensions.

7. The wound cover apparatus of claim 1, wherein the vacuum passageway is a profile channel formed in the vacuum seal.

8. The wound cover apparatus of claim 1, wherein the vacuum passageway comprises intersecting channels.

9. The wound cover apparatus of claim 1, wherein the vacuum seal comprises at least two vacuum passageways which are substantially parallel to one another.

10. The wound cover apparatus of claim 1, wherein the vacuum passageway is a tube comprising a plurality of openings and disposed on the vacuum seal.

11. The wound cover apparatus of claim 1, further comprising:
a seal interface disposed on the vacuum seal, wherein the seal interface comprises a plurality of openings in fluid communication with the vacuum passageway.

12. The wound cover apparatus of claim 1, wherein the treatment enclosure is shaped as a dome.

13. The wound cover apparatus of claim 1, wherein the vacuum seal comprises a flange portion along an outer periphery of vacuum seal.

14. The wound cover apparatus of claim 1, wherein the vacuum seal comprises a valve between the vacuum passageway and the treatment enclosure.

15. The wound cover apparatus of claim 1, wherein the vacuum seal comprises at least one of a thermoplastic polymer and a thermoset polymer.

16. The wound cover apparatus of claim 1, wherein the vacuum seal is flexible.

17. The wound cover apparatus of claim 11, wherein the seal interface comprises at least one of a thermoplastic polymer and a thermoset polymer.

18. The wound cover apparatus of claim 1, further comprising a wound dressing.

19. The wound cover apparatus of claim 18, wherein the wound dressing comprises a therapeutic agent.

20. The wound cover apparatus of claim 18, wherein the wound dressing comprises a polymer and a therapeutic agent.

21. The wound cover apparatus of claim 1, wherein the treatment enclosure and vacuum seal are an integral structure in which the vacuum seal extends outward from the treatment enclosure.

22. The wound cover apparatus of claim 1, wherein the treatment enclosure comprises one or more ridges.

23. The wound cover of claim 14, wherein the valve extends inside the treatment enclosure.

24. A method of medical treatment comprising:
placing a wound cover apparatus onto a user; and
applying negative pressure through the vacuum passageway of a vacuum seal, where the vacuum seal is external to a treatment enclosure of the wound cover apparatus.

25. The method of claim 24, wherein the wound cover apparatus is placed onto the user such that the treatment enclosure is positioned directly above a wound of the user and the vacuum seal is disposed along the periwound.

26. The method of claim 24, further comprising:
applying positive pressure ventilation or negative pressure through an opening of the treatment enclosure while applying a negative pressure through the vacuum passageways of the vacuum seal.

27. The method of claim 24, wherein the negative pressure applied through the vacuum passageway of the vacuum seal is applied intermittently to stimulate biological mechanisms that promote healing.
